(19) United States
(12) Patent Application Publication
(10) Pub. No.: US 2025/0281258 A1
(43) Pub. Date: Sep. 11, 2025

(54) METHOD OF MARKING A CUTANEOUS TISSUE SAMPLE FOR PATHOLOGICAL STUDY

(71) Applicant: SKLIP INC., Lake Oswego, OR (US)

(72) Inventors: Alexander Witkowski, Wesley Chapel, FL (US); Joanna Ludzik, Portland, OR (US)

(21) Appl. No.: 19/074,161

(22) Filed: Mar. 7, 2025

Related U.S. Application Data
(60) Provisional application No. 63/562,281, filed on Mar. 7, 2024.

(51) Int. Cl. A61B 90/00; A61B 5/00; G01N 1/30; G06T 7/00; G06T 11/00; G06T 11/60

(52) U.S. Cl. CPC A61B 90/39; A61B 5/444; G01N 1/30; G06T 7/0012; G06T 11/001; G06T 11/60; A61B 2090/395; G01N 2001/305; G06T 2207/30088; G06T 2207/30096; G06T 2207/30168

(57) ABSTRACT

The method of marking a cutaneous tissue sample for pathological study uses a generated dermoscopy heatmap image in which different colors shown in the heatmap image represent differing degrees of likelihood of malignant or cancerous tissue (e.g., melanoma, basal cell carcinoma or squamous cell carcinoma) or atypical melanocytic nevus of uncertain malignant potential (AMNUMP) (e.g., atypical nevi, spitz nevi, and the like) in a patient's target skin lesion of concern (TSLC) and/or in the patient's pigmented skin lesion of concern (PSLC). The patient's target skin lesion of concern and/or in the patient's pigmented skin lesion of concern is marked in vivo or ex vivo with pigment (e.g., ink, dye, or the like) in areas of interest indicated by the heatmap image. A cutaneous tissue sample is then removed by physical biopsy from the patient.

### CROSS-REFERENCE TO RELATED APPLICATIONS

This application claims the benefit of U.S. Provisional Patent Application No. 63/562,281, filed on Mar. 7, 2024, which is hereby incorporated by reference in its entirety.

### BACKGROUND

### Field

The disclosure of the present patent application relates to dermatological evaluations, and particularly to a method of marking a cutaneous tissue sample for pathological study.

### Description of Related Art

A skin biopsy is a procedure to remove cells from the surface of a patient's body for testing in a lab. A skin biopsy is used most often to diagnose skin conditions and may take a variety of forms. In a shave biopsy, a razor-like tool is used to scrape the surface of the patient's skin. This gathers a cell sample from the top layers of the skin. In a punch biopsy, a round-tipped cutting tool is used to remove a small core of skin, including deeper layers. Excisional biopsies use a scalpel to remove an entire lump or an area of irregular skin. The sample of removed tissue might include a border of healthy skin and the skin's deeper layers.

The skin to be biopsied is cleaned and marked to outline the site. However, the marking, which provides an indication to the medical professional of the region where potentially cancerous tissue is contained, is typically performed purely visually prior to the biopsy procedure. Without additional measurements or tools to define the true boundary which should be marked, such marking may indicate a region which is either too small (thus potentially leaving cancerous tissue remaining) or which is located outside of the true area of concern. Thus, a method of marking a cutaneous tissue sample for pathological study solving the aforementioned problems is desired.

### SUMMARY

The method of marking a cutaneous tissue sample for pathological study records may be used in combination with a method of analyzing digital dermoscopy images, which stores and transfers digital dermoscopy images (DDIs) and provides an initial characterization based on the clinically accepted dermoscopy three-point checklist (D3PC) criteria and the modified three-point checklist (MD3PC) criteria. The initial characterization from the DDIs aids in the clinical characterization of target skin lesions (TSLs) and/or pigmented skin lesions (PSLs) as suspicious for skin cancers (e.g., melanoma, basal cell carcinoma, or squamous cell carcinoma), or atypical melanocytic nevi with uncertain malignant potential (AMNUMP) (e.g., atypical nevi, spitz nevi, etc.), with ternary output to the user. In use, the DDIs may be collected and recorded using a dermatoscope attached to, or otherwise adapted for use with, the digital camera of a mobile device, such as a smartphone, tablet computer or the like.

In the method of marking a cutaneous tissue sample for pathological study, a dermoscopy heatmap image is generated in which different colors shown in the heatmap image represent differing degrees of likelihood of malignant or cancerous tissue (e.g., melanoma, basal cell carcinoma or squamous cell carcinoma) or atypical melanocytic nevus of uncertain malignant potential (AMNUMP) (e.g., atypical nevi, spitz nevi, and the like) in a patient's target skin lesion of concern (TSLC) and/or in the patient's pigmented skin lesion of concern (PSLC). The patient's target skin lesion of concern and/or in the patient's pigmented skin lesion of concern is marked in vivo or ex vivo with pigment (e.g., ink, dye, or the like) in areas of interest indicated by the heatmap image. A cutaneous tissue sample is then removed by physical biopsy from the patient.

As a non-limiting example, in order to generate the dermoscopy heatmap image, a digital dermoscopy image may be received by the mobile device and stored in memory thereof. A determination is made to determine if the digital dermoscopy image meets a quality threshold. This determination is made by software running on the mobile device. If the digital dermoscopy image meets the quality threshold, then a determination is made if the digital dermoscopy image potentially shows a pre-malignant or malignant skin lesion or an atypical melanocytic lesion with uncertain malignant potential. This determination is also made by software running on the mobile device. The determination of the potential showing of the pre-malignant or malignant skin lesion (e.g., melanoma, basal cell carcinoma or squamous cell carcinoma) or the atypical melanocytic lesion with uncertain malignant potential (AMNUMP) is performed using an artificial intelligence system or an augmented intelligence system pre-trained with a dataset of diverse dermoscopy images. The results of the determination are visually indicated to the user on the display of the mobile device.

Further, a digital dermoscopy image heatmap overlay is generated, including an overlay for the DDI with different colors representing differing degrees of likelihood of positive dermoscopy features associated with possible pre-malignant and/or malignant tissue (e.g., melanoma, basal cell carcinoma or squamous cell carcinoma) or atypical melanocytic nevi of uncertain malignant potential (AMNUMP). The digital dermoscopy image heatmap overlay is overlaid on the digital dermoscopy image to make the dermoscopy heatmap image.

The determination of the potential showing of the pre-malignant or malignant skin lesion or the atypical melanocytic nevus of uncertain malignant potential further is performed by feature extraction from the digital dermoscopy image. The features are analyzed by the artificial intelligence system or the augmented intelligence system for the presence of parameters including asymmetry, atypical network, blue-white-grey-violet structures, radial streams, pseudopods, irregular diffuse pigmentation, irregular dots and globules, regression patterns, and combinations thereof. The irregular dots and globules are in the form of round and/or oval structures.

Alternatively, rather than first marking and then performing the biopsy, the ex vivo cutaneous tissue sample may be first removed from the patient by physical biopsy, and then marked (post-biopsy) with pigment (e.g., ink, dye, or the like) in areas of interest indicated by the heatmap image. The marking may be performed immediately post-biopsy (i.e., on the same day) or after receipt in a pathology laboratory (i.e., on the same or a different day). The marked ex vivo cutaneous tissue sample with the pigment may be placed in a fixing solution, such as formalin, as a non-limiting example. Either with or without the use of the dermoscopy heatmap image, a licensed healthcare professional, medical staff, etc. may mark the normal cutaneous tissue surrounding the TSLC/PSLC (included in the biopsy) to mark the anatomic position of the TSLC/PSLC on the patient's body (e.g., directionally north, south, east or west) to provide navigational context (i.e., orientation) to the pathology laboratory team.

These and other features of the present subject matter will become readily apparent upon further review of the following specification.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1A shows a screenshot from a mobile device performing preliminary digital dermoscopy image (DDI) acquisition for use in the method of marking a cutaneous tissue sample for pathological study.

FIG. 1B is a screenshot showing an exemplary “wait” screen while the system analyzes the DDI for quality.

FIG. 2A, FIG. 2B, FIG. 2C, FIG. 2D and FIG. 2E show exemplary screenshots used in the preliminary digital image processing steps of the method of marking a cutaneous tissue sample for pathological study, specifically as used in an educational system.

FIG. 3 is a screenshot showing an exemplary error prompt in the educational system.

FIG. 4A, FIG. 4B and FIG. 4C show exemplary subsequent screenshots used in the preliminary digital image processing steps of the method of marking a cutaneous tissue sample for pathological study, specifically as used in an educational system.

FIG. 5A, FIG. 5B, FIG. 5C, FIG. 5D, FIG. 5E and FIG. 5F show exemplary screenshots used in the preliminary digital image processing steps of the method of marking a cutaneous tissue sample for pathological study, specifically as used in a clinical system.

FIG. 6A is a photograph showing the use of a heatmap to guide placement of pigment as part of the method of marking a cutaneous tissue sample for pathological study.

FIG. 6B is a photograph showing the spraying of a fixing agent following the step illustrated in FIG. 6A.

FIG. 6C shows the presence of pigment fixed to the surface of a TSL and/or PSL of concern (TSLC/PSLC) in vivo.

FIG. 6D shows the presence of the pigment fixed to the surface of the TSLC/PSLC ex vivo and prior to placement into a pathology bottle.

FIG. 7 is a screenshot showing a hematoxylin and eosinophil pathology slide with color ink marking (black arrows) located above the stratum corneum.

Similar reference characters denote corresponding features consistently throughout the attached drawings.

### DETAILED DESCRIPTION

The method of marking a cutaneous tissue sample for pathological study records may be used in combination with a method of analyzing digital dermoscopy images, which stores and transfers digital dermoscopy images (DDIs) and provides an initial characterization based on the clinically accepted dermoscopy three-point checklist (D3PC) criteria and the modified three-point checklist (MD3PC) criteria. The initial characterization from the DDIs aids in the clinical characterization of target skin lesions (TSLs) and/or pigmented skin lesions (PSLs) as suspicious for skin cancers (e.g., melanoma, basal cell carcinoma, or squamous cell carcinoma), or atypical melanocytic nevi with uncertain malignant potential (AMNUMP) (e.g., atypical nevi, spitz nevi, etc.), with ternary output to the user. In use, the DDIs may be collected and recorded using a dermatoscope attached to, or otherwise adapted for use with, the digital camera of a mobile device, such as a smartphone, tablet computer or the like. Non-limiting examples of dermatoscope hardware are shown in U.S. Patent Publication Nos. US 2020/0367803 A1 and US 2022/0370005 A1, each of which is hereby incorporated by reference in its entirety.

In the method of marking a cutaneous tissue sample for pathological study, a dermoscopy heatmap image is generated in which different colors shown in the heatmap image represent differing degrees of likelihood of malignant or cancerous tissue (e.g., melanoma, basal cell carcinoma or squamous cell carcinoma) or atypical melanocytic nevus of uncertain malignant potential (AMNUMP) (e.g., atypical nevi, spitz nevi, and the like) in a patient's target skin lesion of concern (TSLC) and/or in the patient's pigmented skin lesion of concern (PSLC). The patient's target skin lesion of concern and/or in the patient's pigmented skin lesion of concern is marked in vivo or ex vivo with pigment (e.g., ink, dye, or the like) in areas of interest indicated by the heatmap image. A cutaneous tissue sample is then removed by physical biopsy from the patient.

Alternatively, rather than first marking and then performing the biopsy, the ex vivo cutaneous tissue sample may be first removed from the patient by physical biopsy, and then marked (post-biopsy) with pigment (e.g., ink, dye, or the like) in areas of interest indicated by the heatmap image. The marking may be performed immediately post-biopsy (i.e., on the same day) or after receipt in a pathology laboratory (i.e., on the same or a different day). The marked ex vivo cutaneous tissue sample with the pigment may be placed in a fixing solution, such as formalin, as a non-limiting example. Either with or without the use of the dermoscopy heatmap image, a licensed healthcare professional, medical staff, etc. may mark the normal cutaneous tissue surrounding the TSLC/PSLC (included in the biopsy) to mark the anatomic position of the TSLC/PSLC on the patient's body (e.g., directionally north, south, east or west) to provide navigational context (i.e., orientation) to the pathology laboratory team.

As a non-limiting example, in order to generate the dermoscopy heatmap image, a digital dermoscopy image may first be received by the mobile device and stored in memory thereof. A determination is made to determine if the digital dermoscopy image meets a quality threshold. This determination is made by software running on the mobile device. If the digital dermoscopy image meets the quality threshold, then a determination is made if the digital dermoscopy image potentially shows a pre-malignant or malignant skin lesion or an atypical melanocytic lesion with uncertain malignant potential. This determination is also made by software running on the mobile device. The determination of the potential showing of the pre-malignant or malignant skin lesion (e.g., melanoma, basal cell carcinoma or squamous cell carcinoma) or the atypical melanocytic lesion with uncertain malignant potential (AMNUMP) is performed using an artificial intelligence system or an augmented intelligence system pre-trained with a dataset of diverse dermoscopy images. The results of the determination are visually indicated to the user on the display of the mobile device. However, during the initial quality assessment, if the digital dermoscopy image is identified as not being a digital dermoscopy image, or not being of sufficient quality, then the software system operating on the mobile device does not proceed to the next determination step. In this scenario, the output to the user on the display is an error message, such as “ERROR”, for example. If, at the initial determination step, the digital dermoscopy image is confirmed to be a digital dermoscopy image of sufficient quality, then the software proceeds to the second determination step.

A secondary quality test may also be performed before proceeding to the determination of the potential showing of the pre-malignant or malignant skin lesion or the AMNUMP. The digital dermoscopy image may be further evaluated by the software running on the mobile device to determine if the digital dermoscopy image is a) of appropriate technical quality and qualified for the subsequent determination step, or b) not of appropriate technical quality and not qualified for the subsequent determination step. As a non-limiting example, a qualified image may show the imaged target pigmented skin lesion (PSL) is not dry, is not obstructed by hair or other artifacts, and/or is not in a non-qualified anatomical area. If the digital dermoscopy image is not qualified, the software system does not proceed to the subsequent step. In this scenario, the output to the user displays “ERROR” or a similar message. If the digital dermoscopy image is qualified, the software system proceeds to the determination of the potential showing of the pre-malignant or malignant skin lesion or the AMNUMP.

The determination of the potential showing of the pre-malignant or malignant skin lesion or the AMNUMP from the digital dermoscopy image (DDI) is based on a determination if any of the criteria of the D3PC/MD3PC criteria are present in the digital dermoscopy image. If D3PC/MD3PC criteria are not identified (i.e., not present in the DDI), the output to the user on the display of the mobile device may be “NEGATIVE”, as a non-limiting example. For clinical use, a clinical display may be, as a non-limiting example, “UNREMARKABLE”. If at least one D3PC/MD3PC criterion is identified (i.e., present), then the output to the user in an educational system may be, as a non-limiting example, “POSITIVE”, and in a clinical system, may be “SUSPICIOUS”, as a non-limiting example.

It should be understood that any suitable criteria may be used to distinguish the features shown in the DDI. A common method used by licensed healthcare professionals in dermatology, primary care, and other specialties is the dermoscopy three-point checklist (D3PC), as a nonlimiting example, which includes the following: Asymmetry (asymmetry of color and or structure in one or two perpendicular axes); Atypical network (pigment network with irregular holes and thick lines (including round and or oval structures)); and Blue-white structures (any type of blue, white, grey, and or violet color (e.g., a combination of blue-white veil, regression structures, etc.)).

The above may be considered major criteria. Additional nonlimiting minor criteria to consider include: Radial streaming (e.g., streaks, pseudopods, etc.); Irregular diffuse pigmentation (e.g., blotches, dermoscopic islands, etc.); Irregular dots and globules (e.g., round and or oval structures); Any combination of blue, white, grey, and or violet colors suggestive of pigment present in deeper skin layers; and Regression patterns.

Further, a digital dermoscopy image heatmap overlay is generated, including an overlay for the DDI with different colors representing differing degrees of likelihood of positive dermoscopy features associated with possible pre-malignant and/or malignant tissue (e.g., melanoma, basal cell carcinoma or squamous cell carcinoma) or atypical melanocytic nevi of uncertain malignant potential (AMNUMP). The digital dermoscopy image heatmap overlay is overlaid on the digital dermoscopy image to make the dermoscopy heatmap image.

The determination of the potential showing of the pre-malignant or malignant skin lesion or the atypical melanocytic nevus of uncertain malignant potential further is performed by feature extraction from the digital dermoscopy image. The features are analyzed by the artificial intelligence system or the augmented intelligence system for the presence of parameters including asymmetry, atypical network, blue-white-grey-violet structures, radial streams, pseudopods, irregular diffuse pigmentation, irregular dots and globules, regression patterns, and combinations thereof. The irregular dots and globules are in the form of round and/or oval structures.

The method of marking a cutaneous tissue sample for pathological study may be applied to an educational system. The educational system may use the method to analyze target skin lesions (TSLs) and pigmented skin lesions (PSLs) (e.g., moles) on people not restricted by age. The educational system discussed herein is not intended for clinical triage or diagnosis. The clinical decision regarding triage (e.g., to biopsy, monitor, or other patient management) and/or diagnosis of a TSL and/or PSL of concern should be completed independent of the educational system.

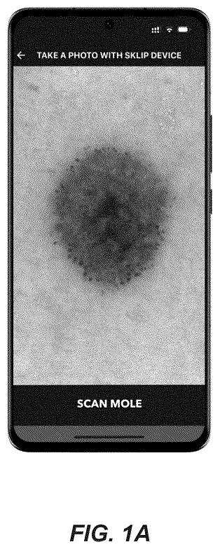

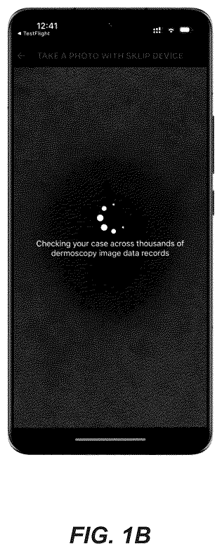

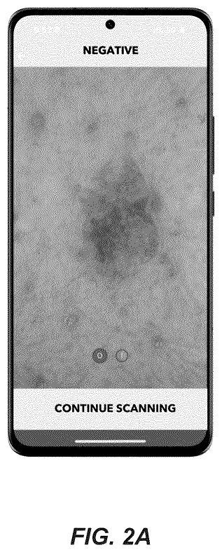

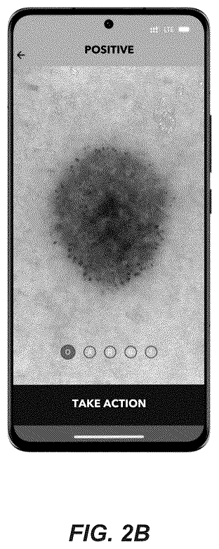

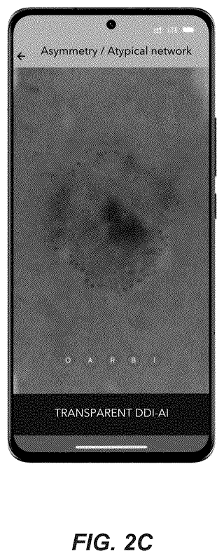

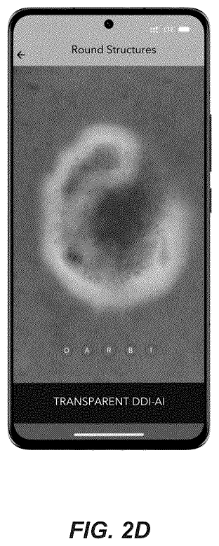

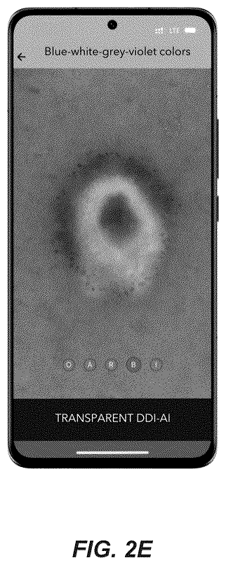

FIG. 1A shows an initial DDI recorded on a mobile device, and FIG. 1B shows an exemplary “wait” screen while the system analyzes the DDI for quality. In FIG. 1A, the user may initiate the determinations may pressing “SCAN MOLE” on the touchscreen display, as a non-limiting example. Assuming that the DDI is of sufficient quality, the educational system provides one of three educational information outputs for each image: “NEGATIVE”; “POSITIVE”; or “ERROR”. “NEGATIVE” indicates limited or no positive D3PC/MD3PC criteria (i.e., no possible immediate concerning dermoscopic features associated with pre-malignancy or malignancy), where one DDI image (the original input DDI) is displayed to the user with information available about the educational information result. FIG. 2A shows an exemplary “NEGATIVE” result.

For the “POSITIVE” output, this indicates positive D3PC/MD3PC criteria (i.e., possible low, moderate, or high concern for pre-malignancy or malignancy). Four DDI graphical digital outputs with information about the educational information result are displayed to the user and can be toggled on the mobile device by swiping the screen left or right, or on a computer by clicking the result window left or right. The first DDI is displayed in its original form with a digital graphic output header text “POSITIVE” (FIG. 2B). The second DDI is displayed with a digital graphic output header text “POSITIVE [Asymmetry/AN]” and a generated digital dermoscopy image heatmap overlay that displays suggested areas within the original input DDI where asymmetry and/or atypical network D3PC/MD3PC umbrella features may be present (FIG. 2C). The third DDI is displayed with a digital graphic output header text “POSITIVE [Round structures],” and a generated digital dermoscopy image heatmap overlay that displays suggested areas within the original input DDI where round and or oval structures D3PC/MD3PC umbrella features may be present (FIG. 2D). The fourth DDI is displayed with a digital graphic output header text “POSITIVE [Blue-white colors or BWGV colors]” and a generated digital dermoscopy image heatmap overlay that displays suggested areas within the original input DDI where blue, white, grey, and or violet color D3PC/MD3PC umbrella features may be present (FIG. 2E). The heatmaps are presented to the user for each individual D3PC/MD3PC feature in three separate images. It should be understood that the term “digital dermoscopy image heatmap,” as used herein, does not refer to a thermogram or thermographic image but rather distinguishes the magnitudes of a particular parameter within the same image by color (similar to how temperature differences and gradients are shown in a thermogram, or how altitudes are shown by color in an altitude map).

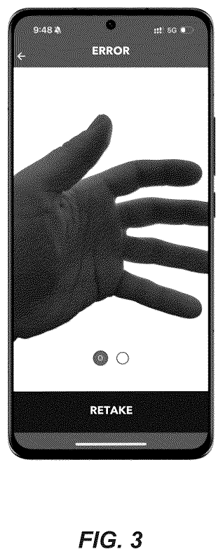

For the “ERROR” output, this indicates that the DDI does not meet the necessary quality criteria or that an assessment of the DDI cannot be made. This result may be returned when the DDI is not of the right type of item (e.g., a DDI taken without dermatoscope hardware) or that the image technical quality is insufficient. The user is then prompted with instructions for how to correctly retake a qualified DDI, as illustrated in FIG. 3.

DDI evaluated with a “POSITIVE” result and having a high likelihood of D3PC/MD3PC positive criteria are marked by the transparent DDI-AI Heatmap overlay with red, orange, and or yellow colors. DDI evaluated by Sklip System Educational with a “POSITIVE” result and have a moderate likelihood of D3PC/MD3PC positive criteria and are marked by the transparent digital dermoscopy image heatmap overlay with teal and/or light blue colors, as non-limiting examples. DDI evaluated with a “POSITIVE” result and having a low likelihood of D3PC/MD3PC positive criteria are marked by the transparent digital dermoscopy image heatmap overlay with dark blue or purple colors, as non-limiting examples.

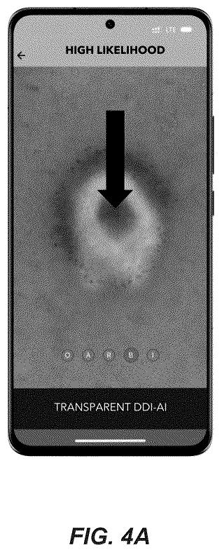

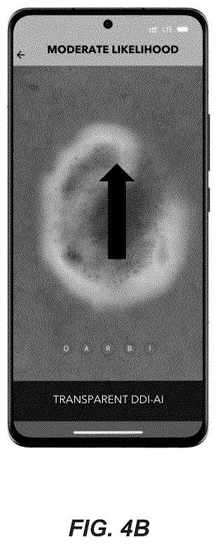

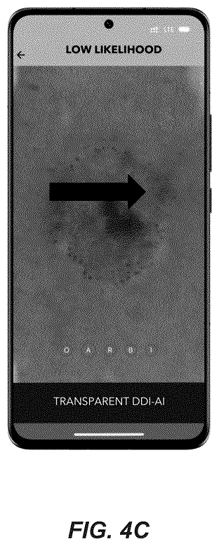

The transparent digital dermoscopy image heatmap overlay provides transparency to the user of what, if any, dermoscopy (dermatoscopy) features are detected by the system. In the non-limiting examples of FIGS. 4A, 4B and 4C, a high likelihood is indicated by a down arrow, a moderate likelihood is indicated by an up arrow, and a low, or absence of, likelihood is indicated by a right arrow.

The method of marking a cutaneous tissue sample for pathological study may also be used with a clinical system. The clinical system is intended for use by licensed healthcare professionals to assess, triage, biopsy or refer a TSL and/or PSL of concern (TSLC/PSLC) to another licensed healthcare professional for a second opinion. A target TSLC/PSLC may be identified by the patient, their partner, family, other persons, or a licensed healthcare professional. Licensed healthcare professionals may include, but are not limited to, medical doctors (MDs), doctors of osteopathy (DOs), nurse practitioners (NPs), physician assistants and or associates (PAs), dental healthcare providers (e.g., DDSs, DMDs, dental hygienists, etc.) who engage in extraoral skin checks, and/or nurses (e.g., RNs, LPNs, etc.) who engage in skin checks and or biopsies. The clinical system may also be used by laypersons in research settings. Layperson use in clinical settings would also require FDA clearance and/or approval. The clinical system is intended for use on adults over 21 years of age.

The clinical system evaluates DDIs for the presence of at least one D3PC/MD3PC feature, and the user interface graphical output is displayed to the user for triage and/or diagnostic purposes. The clinical system is an adjunctive triage and/or diagnostic standalone device and displays one of three possible outputs to the user regarding the target TSLC/PSLC: “SUSPICIOUS,” “UNREMARKABLE,” or “ERROR”. The error message is displayed if the DDI is not able to be assessed or is unreadable. The clinical system is intended to identify dermoscopic (dermatoscopic) features associated with skin cancers (e.g., melanoma, basal cell carcinoma, and squamous cell carcinoma) and atypical melanocytic nevi of uncertain malignant potential (AMNUMP) (e.g., atypical nevi, spitz nevi, etc.). Triage, diagnosis, and patient management decisions are made by the licensed healthcare professional. PSLCs identified by laypersons should be reported to a licensed healthcare professional when a “SUSPICIOUS” result is received and/or the layperson user does not understand the “UNREMARKABLE” or “ERROR” results.

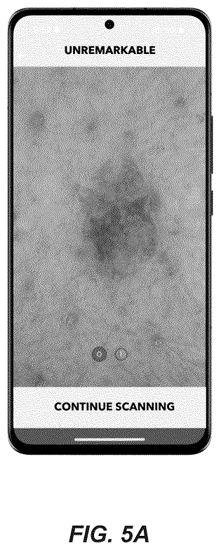

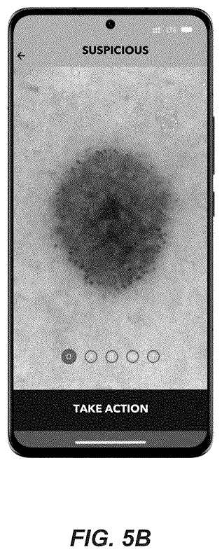

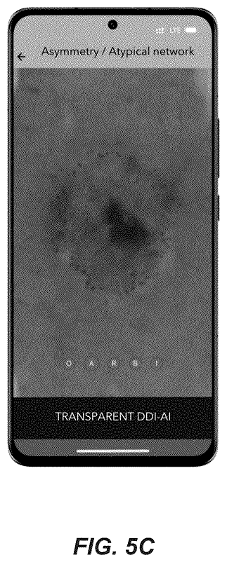

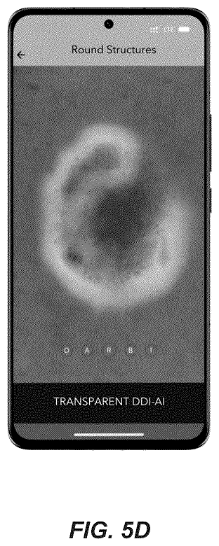

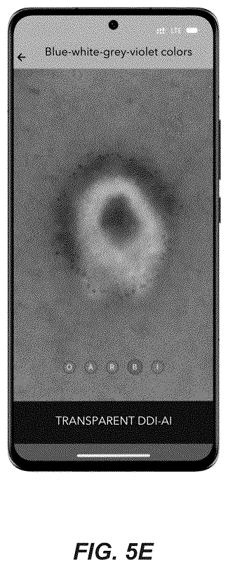

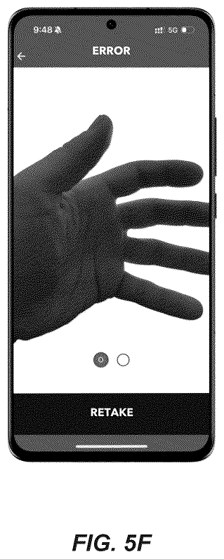

Initially, the user acquires a DDI with appropriate dermoscopic (dermatoscopic) hardware and submits the DDI to the system for evaluation using a similar method as that described above with regard to the educational system. With regard to the “UNREMARKABLE” output, this indicates limited or no positive D3PC/MD3PC features with low concern for pre-malignancy or malignancy. A DDI spot check follow-up within three to six months should be considered. During clinical follow-up, the original DDI may be compared side-by-side with a new DDI of the same PSLC. If there is a change during follow-up by a licensed healthcare professional, then a biopsy should be considered. If there is a change during follow-up by a layperson, then it should be urgently reported to a licensed healthcare professional. One DDI image (the original input DDI) is displayed to the user via graphical output with information available about the clinical result (FIG. 5A).

With regard to a determination and display of “SUSPICIOUS”, this indicates positive D3PC/MD3PC with moderate to high concern for pre-malignancy or malignancy. Additional evaluation is recommended, as determined appropriate by the licensed healthcare professional. This may be a biopsy, or if a biopsy is not taken, then a DDI spot check follow up within three months, or sooner, should be considered. PSLCs identified by laypersons should be reported to a licensed healthcare professional when a “SUSPICIOUS” result is received.

Four DDI are displayed to the user via graphical digital output with information about the clinical result. The first DDI is displayed in its original form with a digital graphic output header with the text “SUSPICIOUS” (FIG. 5B). The second DDI is displayed with a digital graphic output header text “SUSPICIOUS [Asymmetry/AN]” and a generated transparent digital dermoscopy image heatmap overlay that displays suggested areas within the original input DDI where asymmetry and/or atypical network D3PC/MD3PC umbrella features may be present (FIG. 5C). The third DDI is displayed with a digital graphic output header text “SUSPICIOUS [Round structures]” and a generated transparent digital dermoscopy image heatmap overlay that displays suggested areas within the original input DDI where round structures and or oval structure D3PC/MD3PC umbrella features may be present (FIG. 5D). The fourth DDI is displayed with a digital graphic output header text “SUSPICIOUS [Blue-white colors, or BWGV colors]” and a generated transparent digital dermoscopy image heatmap overlay that displays suggested areas within the original input DDI where blue, white, grey, and or violet color D3PC/MD3PC umbrella features may be present (FIG. 5E). The digital dermoscopy image heatmap overlays are presented to the user for each individual D3PC/MD3PC feature in three separate images within the clinical system. It should be understood that the term “heatmap,” as used herein, does not refer to a thermogram or thermographic image but rather distinguishes the magnitudes of a particular parameter within the same image by color (similar to how temperature differences and gradients are shown in a thermogram, or how altitudes are shown by color in an altitude map).

The “ERROR” message is displayed when the DDI does not meet the quality criteria and an assessment of the DDI cannot be made. This result may be returned when the DDI is not of the right sort of item or that the image quality is insufficient. The user is prompted with instructions for how to correctly retake a qualified DDI, as shown in FIG. 5F.

If the clinical system provides an “ERROR” result, the device provides the user with information on how to properly retake a DDI. If the user receives three “ERROR” results in a row for the same target PSLC, the device labelling clearly instructs the user to consult a licensed dermatology healthcare professional. If a licensed healthcare professional receives three “ERROR” results in a row for the same target PSLC, the device labelling clearly instructs the licensed healthcare professional to use their best clinical judgement when choosing PSLC management, independent of the clinical system. If a layperson receives three “ERROR” results in a row for the same target PSLC, the device labelling clearly instructs the layperson to consider reporting their PSLC to a licensed healthcare professional. The use of the clinical system is intended to provide triage and diagnostic clinical assistance. It is not intended to provide a diagnosis that should be made by a licensed pathologist. The clinical system does not provide a specific recommendation for treatment and or management.

The artificial intelligence running on the mobile device was trained by expert dermatologists (dermoscopy experts) who annotated and supervised the algorithm training set using the D3PC/MD3PC to identify dermoscopic features associated with skin cancers (e.g., melanoma, basal cell carcinoma, and squamous cell carcinoma) and atypical melanocytic nevi of uncertain malignant potential (AMNUMP) (e.g., atypical nevi, spitz nevi, etc.). All dermoscopic features of the D3PC/MD3PC are used to interpret DDI that are processed through both the educational and clinical systems. While the use of D3PC versus MD3PC does not influence the output result, or its accuracy, the system's digital graphic outputs prioritize the MD3PC and separates the round and/or oval structures from the atypical network umbrella as an isolated feature for detection and display to the user. This is due to the system's ability to detect round and/or oval structures independent of other dermoscopic criteria with high accuracy. The dermoscopic round and/or oval structures criteria are associated with the highest horizontal growth rate in melanoma skin cancer.

Table 1 below shows the dermoscopic (dermatoscopic) criteria used to train the AI system (showing no difference in evaluation criteria between D3PC and MD3PC) versus the system's user interface (UI) output that includes atypical network in the asymmetry group and places round and or oval structures an independent feature for display to the user.

TABLE 1

| Dermoscopy three- | Modified dermoscopy | User interface (UI) |
|---|---|---|
| point checklist | three-point checklist | output when DDI is |
| (D3PC) used to | (MD3PC) used to | positive for at least one |
| evaluate DDI | evaluate DDI | D3PC/MD3PC feature |
| Asymmetry | Asymmetry and/or | Asymmetry and/or |
| (geometric/axial) | atypical network | atypical network |
| Atypical network | Round structures | Round structures |
| (includes round and/ | (including oval | |
| or oval structures) | structures) | |
| Blue-white structures | Blue-white-grey- | Blue-white-grey- |
| (includes grey and | violet colors | violet colors |
| violet colors) | | |

As discussed above, in addition to the ternary outputs (e.g., “NEGATIVE,” “POSITIVE,” and “ERROR” for the educational system and “UNREMARKABLE,” “SUSPICIOUS,” and “ERROR” for the clinical system), the device produces an additional output when the DDI input is classified as “POSITIVE” (educational) or “SUSPICIOUS” (clinical). This additional output is a generated transparent digital dermoscopy image heatmap overlay showing where possible positive D3PC/MD3PC features are located within the DDI on an X-Y axis, or longitude-latitude axis, where the reference map is the original input DDI. The heatmap images are presented to the user for each individual D3PC/MD3PC feature in three separate images.

DDI with high likelihood of D3PC/MD3PC positive criteria are marked with red, orange, and/or yellow colors. DDI with moderate likelihood of D3PC/MD3PC positive criteria are marked with teal or light blue colors. DDI with low likelihood, or possible absence, of D3PC/MD3PC positive criteria are marked with dark blue or purple colors.

A licensed healthcare professional, medical staff, or the like may acquire the digital dermoscopy image (DDI) of a target skin lesion of concern or pigmented skin lesion of concern (TSLC/PSLC) already deemed for biopsy and run it through the artificial intelligence (AI) or augmented intelligence (AUI) discussed above, as well as uploading the DDI and/or the associated reports (digital graphic outputs) into a mobile phone app or an electronic patient medical record (EMR). The generated transparent heatmap overlay may be used by a licensed healthcare professional, medical staff or the like as a guide for marking the skin tissue in vivo (“in vivo ink marking”) prior to a physical biopsy (e.g., shave, deep shave, punch, tangential, excision, or the like), or just after a physical biopsy but prior to placement of ex vivo biopsied tissue material into a fixing substance (e.g., formalin or the like), with tissue marking ink (or dye) of any color spectrum, or other suitable material, with or without a fixing agent (e.g., vinegar or the like), on the surface of the TSLC/PSLC in areas highlighted by the heatmap. It should be understood that the heatmap may include, or be appended with, any suitable symbols, colors or the like to delineate the positive dermoscopic (dermatoscopic) criteria (e.g., dermoscopy three-point checklist, modified dermoscopy three-point checklist, or the like) present within the DDI (e.g., red, orange, yellow, or other colors).

The licensed healthcare professional, medical staff, or the like may then complete a physical biopsy (e.g., shave, deep shave, punch, tangential, excision, or other) of the TSLC/PSLC after dye (or ink) marking to create an ex vivo tissue material sample. The ex vivo tissue sample (e.g., with the ink marking on the surface created in vivo, ink marking added the same day post-physical biopsy in the healthcare professional office ex vivo, etc.) may then be submitted to a pathology laboratory for technical slide preparation and/or evaluation by a licensed pathologist (or dermatopathologist). Alternatively, with an accompanying DDI and with or without the use of the heatmap, the licensed healthcare professional, medical staff or the like may mark the normal cutaneous tissue surrounding the TSLC/PSLC (that is included in a biopsy) to mark the anatomic position of the TSLC/PSLC on the patient's body (e.g., directionally north, south, east or west) to provide navigational context (orientation) to the pathology laboratory team.

Alternatively, the licensed healthcare professional, medical staff or the like may acquire heatmap outputs and may choose not to mark the in vivo or ex vivo tissue sample with pigment using ink (or dye). Instead, the ex vivo tissue sample may be evaluated later by a pathology laboratory team (e.g., laboratory technician, laboratory tissue grosser, laboratory histotech, pathologist, dermatopathologist, or the like) with dermatoscope hardware, DDI, and or the analyzed DDI, as discussed above, after which the pathology laboratory team may mark the ex vivo tissue sample post receipt with dye (or ink) marking at the pathology laboratory, guided by the heatmaps.

It is noted that the above ink (or dye) marking and biopsy steps assume that a “POSITIVE” or “SUSPICIOUS” result output was obtained from the educational or clinical system. If a “NEGATIVE” or “UNREMARKABLE” output was obtained, the licensed healthcare professional should use their clinical judgment to triage, diagnose and/or manage the TSLC/PSLC, independent of the system output. For the avoidance of doubt, the system used does not have to be limited to the educational and clinical systems described above but, instead, may include any suitable device or system having similar features and functionality such that it meets all the requirements discussed above.

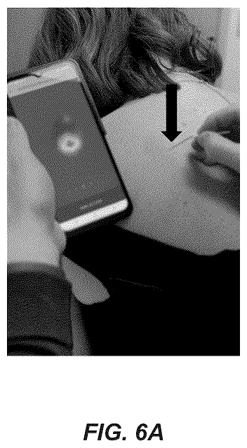

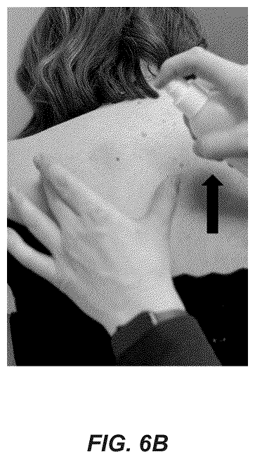

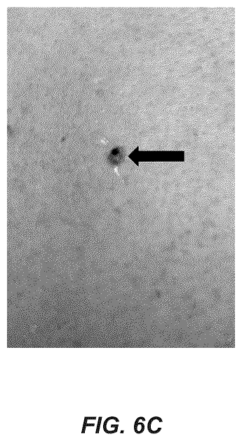

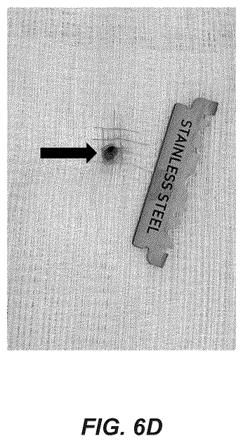

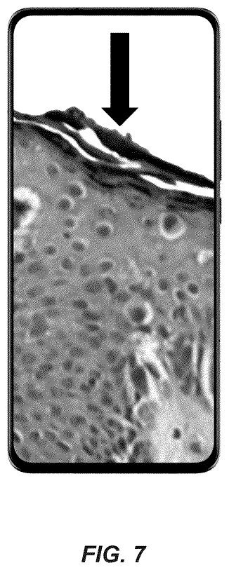

FIG. 6A shows the use of the heatmap to guide placement of ink (or dye) with an object with a sharp or round ending (indicated in FIG. 6A with a down arrow). FIG. 6B illustrates the spraying a fixing agent (e.g., vinegar) (indicated in FIG. 6B with an up arrow). FIG. 6C shows the presence of the ink (or dye) fixed to the surface of the TSLC/PSLC in vivo (indicated in FIG. 6C with a left arrow). FIG. 6D shows the presence of the ink (or dye) fixed to the surface of the TSLC/PSLC ex vivo and prior to placement into a pathology bottle (indicated in FIG. 6D with a right arrow).

The pathology laboratory team (e.g., laboratory technician, laboratory tissue grosser, laboratory histotech, pathologist, dermatopathologist or the like) may review the “POSITIVE” or “SUSPICIOUS” outputs, including the heatmaps. This review of the associated ex vivo tissue material sample may be performed on a mobile device (e.g., smartphone, tablet, or the like), computer (e.g. desktop, laptop, or the like), or a printout (e.g., a physical pathology request form on paper, or the like) for the purpose of tissue orientation and context of the presence or absence of dermoscopic (dermatoscopic) features.

The pathology laboratory team, as described above, may first consider using current standard practice(s) for laboratory ex vivo tissue processing methods to process and prepare the tissue sample, pathology block(s), and or slide(s) for review, evaluation, and or diagnosis by a licensed pathologist or dermatopathologist (i.e., “standard practice”). After consideration to first use standard practice, the laboratory team may consider preparing an additional technical pathology tissue sample, block, and or slide at the site(s) of the ink (dye) marking (“Ink Focused Pathology Slide Preparation”) to ensure the pathology slides prepared from the ex vivo tissue material sample are representative of the dermoscopic (dermatoscopic) features within the TSLC/PSLC surface area; i.e., the original reason for biopsy. This is in comparison to standard practice where a traditional pathology request form that contains protected health information (PHI), which rarely contains a clinical photo (not a DDI), and a written differential diagnosis by the submitting licensed healthcare professional who completed the physical biopsy. The digital outputs described above provide additional context to a licensed pathologists' (or dermatopathologists') Pathology Request in an effort to enhance diagnostic precision by adding objective datapoints, thus enabling reduction of TSLC/PSLC ex vivo cutaneous tissue sampling bias (e.g., misdiagnosis, delayed diagnosis, inter-operator error, inter-operator pathology variability, and the like) that is prevalent in Standard Practice.

A licensed pathologist, dermatopathologist or the like may then use a digital device (e.g., smartphone, tablet, computer, or the like) to review digital output discussed above, including the heatmaps, and then complete a focused review across all TSLC/PSLC skin layers of the pathology slides (“DDI-AI Ink Focused Review”) that contain visible ink (or dye) marking at the superficial aspect (above the stratum corneum) in their consideration of the TSLC/PSLC differential and final diagnosis (e.g., melanoma, basal cell carcinoma, squamous cell carcinoma, atypical melanocytic nevi of uncertain malignant potential, or the like). DDI-AI Ink Focused Review may be completed with or without the assessment of skin layers of pathology slide(s) prepared using standard practice block and slide technical processing and preparation methods which may contain TSLC/PSLC tissue sampling bias (e.g., misdiagnosis, delayed diagnosis, inter-operator error, inter-operator pathology variability or the like) that result in pathology tissue (or slides) presented to a pathologist (or dermatopathologist) not being representative of areas within the TSLC/PSLC that prompted the licensed healthcare professional to complete the original physical biopsy.

Alternatively, the pathology laboratory team (e.g., laboratory technician, laboratory tissue grosser, laboratory histotech, pathologist, dermatopathologist or the like) may choose not to acquire the digital output and/or heatmap review, but instead proceed with the Ink Focused Pathology Slide Preparation and or the Ink Focused Review and complete the final diagnosis (based on Ink Focused Review and review of other area samples within the ex vivo tissue material sample per standard practice). As a further alternative, the pathology laboratory team may receive an ex vivo tissue material sample with no ink marking and issue a final diagnosis based on standard practice.

The above allows a licensed healthcare professional to use ink (or dye) marking based on the heatmap to communicate to the pathology laboratory team (e.g., laboratory technician, laboratory tissue grosser, laboratory histotech, pathologist, dermatopathologist or the like) where to complete ex vivo tissue material sample sectioning to process, prepare, and create a pathology block(s) and/or pathology slide(s) to reduce sampling bias via ink (or dye) focused pathology slide preparation. This may help a licensed healthcare professional communicate objectively to a pathologist, or dermatopathologist, why the TSLC/PSLC was selected for biopsy and improve correct diagnosis of TSLCs/PSLCs that are in differential diagnosis with skin cancers (melanoma, basal cell carcinoma, squamous cell carcinoma, atypical melanocytic nevi of uncertain malignant or the like).

An exemplary image (that can be viewed on a physical pathology slide, paper printout, digital mobile device, computer, or the like) showing a hematoxylin and eosinophil (H&E) pathology slide with green color ink marking (black arrows) located above the stratum corneum is provided in FIG. 6.

It is to be understood that the method of marking a cutaneous tissue sample for pathological study is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A method of marking a cutaneous tissue sample for pathological study, comprising:
generating a dermoscopy heatmap image, wherein different colors shown in the heatmap image represent differing degrees of likelihood of malignant or cancerous tissue or atypical melanocytic nevus of uncertain malignant potential in a patient's target skin lesion of concern and/or in the patient's pigmented skin lesion of concern;
marking the patient's target skin lesion of concern and/or in the patient's pigmented skin lesion of concern in vivo or ex vivo with pigment in areas of interest indicated by the heatmap image; and
removing by physical biopsy a cutaneous tissue sample from the patient.

2. The method of marking a cutaneous tissue sample for pathological study as recited in claim 1, wherein the generation of the dermoscopy heatmap image comprises:
receiving a digital dermoscopy image;
determining if the digital dermoscopy image meets a quality threshold;
when the digital dermoscopy image meets the quality threshold, determining if the digital dermoscopy image potentially shows a pre-malignant or malignant skin lesion or an atypical melanocytic lesion with uncertain malignant potential, wherein the determination of the potential showing of the pre-malignant or malignant skin lesion or the atypical melanocytic lesion with uncertain malignant potential is performed by an artificial intelligence system or an augmented intelligence system pre-trained with a dataset of diverse dermoscopy images;
visually indicating to a user results of the determination;
generating a digital dermoscopy image heatmap overlay containing the different colors; and
overlaying the digital dermoscopy image heatmap overlay on the digital dermoscopy image.

3. The method of marking a cutaneous tissue sample for pathological study as recited in claim 2, wherein the determination of the potential showing of the pre-malignant or malignant skin lesion or the atypical melanocytic nevus of uncertain malignant potential further comprises feature extraction from the digital dermoscopy image, wherein features are analyzed by the artificial intelligence system or the augmented intelligence system for a presence of parameters selected from the group consisting of asymmetry, atypical network, blue-white-grey-violet structures, radial streams, pseudopods, irregular diffuse pigmentation, irregular dots and globules, regression patterns, and combinations thereof.

4. The method of marking a cutaneous tissue sample for pathological study as recited in claim 3, wherein the irregular dots and globules comprise round and/or oval structures.

5. A method of marking a cutaneous tissue sample for pathological study, comprising:
generating a dermoscopy heatmap image, wherein different colors shown in the heatmap image represent differing degrees of likelihood of malignant or cancerous tissue or atypical melanocytic nevus of uncertain malignant potential in a patient's target skin lesion of concern and/or in the patient's pigmented skin lesion of concern;
removing by physical biopsy an ex vivo cutaneous tissue sample from the patient;
marking the ex vivo cutaneous tissue sample with pigment in areas of interest indicated by the heatmap image.

6. The method of marking a cutaneous tissue sample for pathological study as recited in claim 5, further comprising placing the marked ex vivo cutaneous tissue sample with the pigment in a fixing solution.

7. The method of marking a cutaneous tissue sample for pathological study as recited in claim 6, wherein the fixing solution comprises formalin.

8. The method of marking a cutaneous tissue sample for pathological study as recited in claim 5, further comprising marking normal cutaneous tissue surrounding the patient's target skin lesion of concern and/or the patient's pigmented skin lesion of concern to mark an anatomic position of the patient's target skin lesion of concern and/or the patient's pigmented skin lesion of concern on the patient's body.

9. The method of marking a cutaneous tissue sample for pathological study as recited in claim 5, wherein the generation of the dermoscopy heatmap image comprises:
receiving a digital dermoscopy image;
determining if the digital dermoscopy image meets a quality threshold;
when the digital dermoscopy image meets the quality threshold, determining if the digital dermoscopy image potentially shows a pre-malignant or malignant skin lesion or an atypical melanocytic lesion with uncertain malignant potential, wherein the determination of the potential showing of the pre-malignant or malignant skin lesion or the atypical melanocytic lesion with uncertain malignant potential is performed by an artificial intelligence system or an augmented intelligence system pre-trained with a dataset of diverse dermoscopy images;
visually indicating to a user results of the determination;
generating a digital dermoscopy image heatmap overlay containing the different colors; and
overlaying the digital dermoscopy image heatmap overlay on the digital dermoscopy image.

10. The method of marking a cutaneous tissue sample for pathological study as recited in claim 9, wherein the determination of the potential showing of the pre-malignant or malignant skin lesion or the atypical melanocytic nevus of uncertain malignant potential further comprises feature extraction from the digital dermoscopy image, wherein features are analyzed by the artificial intelligence system or the augmented intelligence system for a presence of parameters selected from the group consisting of asymmetry, atypical network, blue-white-grey-violet structures, radial streams, pseudopods, irregular diffuse pigmentation, irregular dots and globules, regression patterns, and combinations thereof.

11. The method of marking a cutaneous tissue sample for pathological study as recited in claim 10, wherein the irregular dots and globules comprise round and/or oval structures.
